SEQUENCE LISTING (4 sequences)

SEQ ID NO: 1 - protein, 478 aa, Homo sapiens
MSTNGDDHQVKDSLEQLRCHFTWELSIDDDEMPDLENRVLDQIEFLDTKYSVGIHNLLAYVKHLKGQNEEALKSLKEAENLMQEEHDNQANVRSLVTWGNFAWMYYHMGRLAEAQTYLDKVENICKKLSNPFRYRMECPEIDCEEGWALLKCGGKNYERAKACFEKVLEVDPENPESSAGYAISAYRLDGFKLATKNHKPFSLLPLRQAVRLNPDNGYIKVLLALKLQDEGQEAEGEKYIEEALANMSSQTYVFRYAAKFYRRKGSVDKALELLKKALQETPTSVLLHHQIGLCYKAQMIQIKEATKGQPRGQNREKLDKMIRSAIFHFESAVEKKPTFEVAHLDLARMYIEAGNHRKAEENFQKLLCMKPVVEETMQDIHFHYGRFQEFQKKSDVNAIIHYLKAIKIEQASLTRDKSINSLKKLVLRKLRRKALDLESLSLLGFVYKLEGNMNEALEYYERALRLAADFENSVRQGP

SEQ ID NO: 2 - protein, 482 aa, Homo sapiens
MSEIRKDTLKAILLELECHFTWNLLKEDIDLFEVEDTIGQQLEFLTTKSRLALYNLLAYVKHLKGQNKDALECLEQAEEIIQQEHSDKEEVRSLVTWGNYAWVYYHMDQLEEAQKYTGKIGNVCKKLSSPSNYKLECPETDCEKGWALLKFGGKYYQKAKAAFEKALEVEPDNPEFNIGYAITVYRLDDSDREGSVKSFSLGPLRKAVTLNPDNSYIKVFLALKLQDVHAEAEGEKYIEEILDQISSQPYVLRYAAKFYRRKNSWNKALELLKKALEVTPTSSFLHHQMGLCYRAQMIQIKKATHNRPKGKDKLKVDELISSAIFHFKAAMERDSMFAFAYTDLANMYAEGGQYSNAEDIFRKALRLENITDDHKHQIHYHYGRFQEFHRKSENTAIHHYLEALKVKDRSPLRTKLTSALKKLSTKRLCHNALDVQSLSALGFVYKLEGEKRQAAEYYEKAQKIDPENAEFLTALCELRLSI

SEQ ID NO: 3 - protein, 508 aa, Rabbit
MWNPQRTRARSQRAQSGSGNLQTSASFSATMSECAEEHPLKDRLQKLRCHFTWGLLIEDTGLPDLEDRILEEIQFLDTENKVGYNLLAYVKHLQGKHEDALENLKEAEEVVQGDQADHSDVRSLVTWGNYAWVHYHMGRLADAQTYLDKVENTCQKSADPTRYSTQCPEMDCEEGWALLKCGGKNYERAKACFEKALEADPENPEFNTGYAITVYRLDYPAKRPCDVSDAFSLQPLRKAIRLNPQDAYLKALLALKLQDVGEEAEGRECLEEALAHTSSQTYVFRYAAKFFRRQGRVDEALKYLKMALKATPSSAFLHQQIGLCYKKKTNQIMNATHMQPTRQDRENVDRLIQLAIFHFEYAVKQKPTFEVAYVDLARMYITAGDHEKAEDTFQKVLCMTPLQEHIQQNIHFSYGQFQQFQKKSEVDAITHYLQAVTIRKDSYARDKSIKALEQLVSWKLERNPLDQEALSLREVLHRLVGGRDEALECSEQDLRLAADSGNWVGSSL

SEQ ID NO: 4 - protein, 482 aa, Danio rerio
MTSDVSSMEADRALRTKLHQLECHFTWALIKDDIDINDLLNRLEEQINLDLEKKERLARTYSALAYVQYLLGFHEKAHQSLMTSKKLHIESHGDEFYRTLIVTYGNLAWLNYHMKNYTECESYLNSLQRINETSPAEFSSIPEVLGEKGWTFLKFSRKYYDGAKECFRKAVELEPEEPEWHTGYAIALYRTEFESTVLEDSATVKQLRLAIEMNPDDDVLKVLLSLRLIVYKRYGEAESWVEKALEKSPDHPHVMRYVGKFFRNKGCVDRSIDLLKRALERSPNSSFIHHQLALCYKYKKIQVLQEQSHHARGSRVQQLRDQCIFHLEKATSLTTSFISAMSDLALQYGENGDIPRAEELFQVTFKIAKEKNDGLHVVNYYYAEYQLYCHRCEPLAVQHYMECLKMCPKSVEGRISSTRMKKTAEKWIDRKSQEGKAYGMLAFLHKVKGEIAQAIECYEKALSYEDNNEFLRNLRELRLSLL

(19) United States
(12) Patent Application Publication
(10) Pub. No.: US 2022/0251546 A1
(43) Pub. Date: Aug. 11, 2022

(54) METHOD FOR RNA SELECTION AND/OR ENRICHMENT, RNA MOLECULE AND USE THEREOF

(71) Applicant: UNIWERSYTET WARSZAWSKI, Warszawa (PL)

(72) Inventors: Maria Gorna, Warszawa (PL); Martyna Nowacka, Warszawa (PL); Matylda Izert, Warszawa (PL); Monika Kowalska, Zabki (PL); Natalia Karolak, Warszawa (PL); Maria Klimecka, Warszawa (PL); Krzysztof Mlynarczyk, Kielce (PL)

(21) Appl. No.: 17/616,459

(22) PCT Filed: Jun. 4, 2020
(86) PCT No.: PCT/IB2020/055263

§ 371 Date: Dec. 3, 2021

(30) Foreign Application Priority Data
Jun. 5, 2019 (PL) PL43015 1

(51) Int. Cl. C12N 15/10; C12Q 1/6806

(52) U.S. Cl. CPC C12N 15/1068; C12Q 1/6806

(57) ABSTRACT

A method for RNA selection and/or enrichment, especially with mRNA molecules, from a pool of RNA molecules, the method comprising a step of incubating a sample containing a pool of RNA molecules with an RNA binding protein to form RNA-RNA binding protein complexes, wherein a protein of IFIT family of proteins or its functional variants, homologues or mutants are used as the RNA binding protein. The RNA molecule selected and/or enriched by the method and is used for detection in RNA pathogen-based diagnostic tests and for preparation of libraries for RNA sequencing.

The subject of the invention is a method for RNA selection and/or enrichment, especially with mRNA molecules, from a pool of RNA molecules, an RNA molecule selected and/or enriched by this method, as well as its use.

The new RNA selection and/or enrichment method of the invention is used in molecular analysis, sequencing and diagnostics. For example, the RNA selection and enrichment can be used to prepare libraries for high-throughput RNA sequencing (RNA-Seq) or to enrich RNA material for detection in pathogen RNA-based diagnostic tests.

Approximately 80% of the total RNA in eukaryotic cells is rRNA, a further 15% is tRNA. In prokaryotic cells, rRNA and tRNA account for >97% of total RNA. In both cases, the mRNA and other non-coding RNA molecules constitute less than 5% of the total RNA.

Molecular analyzes, such as, for example, high-throughput RNA sequencing or nucleic acid-based diagnostic analyses, in which the tested object is exclusively a part of the transcriptome that encodes proteins, therefore require the enrichment of the total RNA sample with the messenger RNA (mRNA) molecules. The stage of RNA sample enrichment with mRNA is crucial for the efficiency of molecular analyses.

High-throughput RNA sequencing, RNA-Seq, is currently one of the main methods of transcriptome testing, including gene expression analysis, testing alternative mRNA splicing, and post-transcriptional modifications. Preparation of cDNA libraries for RNA-Seq, in particular for next generation sequencing (NGS), is, however, adapted to the part of the transcriptome, which is to be analyzed. Where only the protein-coding part of a transcriptome is tested, the total RNA sample should be selected to remove high-copy RNAs (rRNA and tRNA). This step is necessary to get a good quality sequencing result.

Known methods for enriching an RNA sample with coding sequences include, but are not limited to, removing rRNA using commercial kits, such as, for example, RiboMinus™ (ThermoFisher Scientific, Invitrogen), which use species-specific probes to selectively capture ribosomal RNA sequences. Other commercially available kits, such as NEBNext® rRNA Depletion Kit (New England Biolabs) or Ribo-Zero™ rRNA Removal Kits (Epicenter, Illumina) are also used for rRNA removal. However, these sets have their limitations. For example, RiboMinus (Invitrogen) kits contain LNA (Locked Nucleic Acid) probes designed to selectively and efficiently remove rRNA from the target organism. Popular commercial kits offer probes compatible with RNA isolated from human, mouse, rat, yeast and bacteria cells. While they can be used for total RNA samples isolated from organisms other than those to which the kit is dedicated, the efficiency of rRNA depletion is then significantly reduced. Getting rid of rRNA from samples isolated from organisms that are not model ones is therefore difficult, lengthy and expensive, as it requires several successive depletion rounds. Among other things, the scientific literature describes significant differences in the efficiency of rRNA depletion depending on which of the dedicated commercial kits was used (0.5% to 75% of the mapping sequences for rRNA remained in the library, Petrova et al. (2017), Scientific Reports, 7 (41114)).

In addition to the above methods, rRNA and tRNA removal is carried out using exoribonucleases such as Terminator™ 5′-Phosphate Dependent Exonuclease, which selectively degrades molecules with a monophosphate group at the 5′ end or with RNase H and oligonucleotides complementary to rRNA sequences.

Some of the currently used RNA selection methods are based on the fact that mRNA molecules may have various types of modifications at the 3′ end and 5′ end. In the case of mRNAs of eukaryotic organisms, most of the mRNA molecules are polyadenylated by adding a series of adenosine at the 3′ end (polyA tail). A characteristic modification of the mRNA 5′ end in eukaryotic organisms is the presence of so-called cap, i.e. a modified nucleoside, 7-methylguanosine, 5′ m7G, attached with a 5′-5′ triphosphate bond to the first nucleotide of the RNA molecule at the 5′ end. Depending on the RNA origin, different types of 5′ end modifications can be distinguished, such as, for example, Cap 0, Cap 1, Cap 2. The presence of Cap 0 is characteristic for mRNA of lower eukaryotic organisms and plants, while Cap 1 and Cap 2 have additional modifications and occur in mRNA of higher eukaryotes. Prokaryotic mRNA contains a triphosphate group at the 5′ end.

One of the methods based on modification of the molecule end is the RNA selection and enrichment method based on the selective uptake of mRNA molecules containing a polyA tail at the 3′ end. Enrichment of the test sample with RNA molecules polyadenylated at the 3′ end can be performed using commercially available kits, such as, for example, NEBNext® Poly(A) mRNA Magnetic Isolation Module (New England Biolabs), Dynabeads™ mRNA Purification Kit (Invitrogen) or Seq-Star™ poly(A) mRNA Isolation Kit (Arraystar Inc). However, this method has its limitations. Performing polyA selection does not guarantee removal of high-copy RNA molecules from the test sample. As a result, the sample may still contain large amounts of rRNA, which due to the presence of internal segments of polyA sequence repeats may be enriched with the mRNA fraction. Due to the fact that polyadenylation of the 3′ end concerns only mRNA from eukaryotic cells, it is a method not applicable to samples isolated from bacteria. It should also be noted that not all eukaryotic mRNAs are polyadenylated. PolyA tails are absent e.g. in mRNAs of histone proteins, for which this method is not applicable either.

In the publication of Blower et al. (2013), PLoS ONE 8 (10): e77700 eIF4E protein was used, which binds to mRNA with the Cap 0 structure at the 5′ end. The use of eIF4E to bind mRNA to the cap is quite obvious as it is a protein well-known for its involvement in the initiation process of translation. However, this method has its limitations—it requires methylation at the 7G position (nucleotide at the cap end) for full binding efficiency. In case the methylation is not present or has been lost (by chemical degradation or enzymatic modifications), eIF4E will not recognize such RNA. eIF4E does not distinguish 2′O methylation at the first nucleotides, i.e. it does not distinguish Cap 0 from Cap 1 and Cap 2, and thus it does not distinguish between RNA of lower eukaryotes and RNA of higher eukaryotes. In addition, it does not recognize ppp at the 5′ end of RNA, and therefore does not apply to samples isolated from bacteria. Furthermore, the eIF4E protein encoding gene is constitutively expressed in cells and the protein is present in cells under normal conditions, so the method involves the risk of artifacts arising from the interaction of cellular proteins with the eIF4E.

Although there are currently many methods for selecting and enriching the RNA sample with coding sequences, all these methods have their limitations, are not efficient enough, often limited only to dedicated species, and also lengthy and costly because they require many selection rounds.

There is therefore a need to develop a method for selecting and enriching the RNA sample with the desired type of molecules, especially with mRNA, for applications in molecular biology and diagnostics, that would be efficient, versatile in species, and also relatively easy and quick to perform, and thus economically advantageous.

The inventors have surprisingly found that for selection and enrichment of the RNA sample, for applications in molecular biology, it is possible to use proteins from the IFIT protein family to obtain a method that is universal, fast, based on simple laboratory techniques and also economically advantageous.

IFIT proteins (Interferon-Induced Proteins with Tetratricopeptide Repeats) are produced in a human body in response to a viral infection. Genes encoding IFIT proteins are expressed in interferon-induced inflammation caused by a viral pathogen. Thus, IFIT proteins belong to the innate immune system and their role is to detect and block the viral RNA translation, thus inhibiting the multiplication of the virus in cells. Four human IFIT proteins have been characterized so far, including IFIT1 (also known as ISG56), IFIT2 (ISG54), IFIT3 (ISG60) and IFIT5 (ISG58).

It has surprisingly been found that IFIT proteins can be successfully used in the RNA selection and enrichment method for molecular biology applications of the invention. While viral RNA is the natural target of IFIT proteins activity in a human body, the method according to the invention based on IFIT proteins goes far beyond natural or artificial RNA ligands and proposes the use of IFIT proteins intentionally to select and enrich other RNAs, also from organisms such as bacteria or lower eukaryotes, such as yeast, which are not the natural targets of IFIT activity.

The subject of the invention is a method for RNA selection and/or enrichment, especially in mRNA molecules, from a pool of RNA molecules, the method comprising a step of incubating a sample containing a pool of RNA molecules with an RNA binding protein to form RNA-RNA binding protein complexes, said method being characterized by the fact that a protein of the IFIT protein family or its functional variants, homologues or mutants are used as the RNA binding protein.

Preferably, in the method of the invention, the protein of the IFIT protein family is a protein containing a tetratricopeptide repeat (TPR) region and a structural motif with an amino acid sequence CHFxW, where x means T or N or another amino acid present in a helical twist in a loop between alpha-helices. The TPR repeat region as well as the CHFxW motif are characteristic of the IFIT protein family, with the CHFxW motif being one of the most conserved motifs among the IFIT homologs, which is known to be essential to maintain the protein structure (mutations within this motif cause protein instability).

Preferably, the protein of the IFIT protein family is a protein comprising an amino acid sequence which is at least 20% identical to the amino acid sequence of the human IFIT1 protein shown as SEQ ID NO: 1, or a functional fragment thereof. Equally preferably, in the method of the invention, the protein of the IFIT protein family is a protein comprising an amino acid sequence which is at least 20% identical to the amino acid sequence of the human IFIT5 protein shown as SEQ ID NO: 2, or a functional fragment thereof.

Most preferably, in the method of the invention, the protein of the IFIT protein family is a human IFIT1 protein with the amino acid sequence shown as SEQ ID NO: 1 and/or a human IFIT5 protein with the amino acid sequence shown as SEQ ID NO: 2, or mutants thereof.

Preferably, in the method of the invention, the protein of the IFIT protein family is a functional variant of the IFIT1 protein and/or the IFIT5 protein from a non-human organism, or mutants thereof.

In the method of the invention, it is preferred that the IFIT1 protein and/or IFIT5 protein is a recombinant protein which may additionally contain an oligohistidine tag, a Strep-tag, a One-Strep or other elements for purifying or immobilizing the protein and optionally a protease cleavage site between the tag and the tag removal protein, and optionally the SUMO tag or other domains improving the protein solubility or stability.

The recombinant IFIT1 and/or IFIT5 protein can be used, according to the invention, in free form or, optionally, in a form associated with a reporter molecule, such as a tagged RNA molecule that competes with a RNA molecule pool for binding to the protein IFIT1 and/or IFIT5. If a sample containing a RNA molecule pool contains RNA molecules, which the IFIT protein binds better than the reporter molecule, the reporter molecule is released and the target RNA molecule is bound. Then the presence of a free reporter molecule will indicate the presence of the RNA of interest in the sample. A released reporter molecule, such as a tagged RNA molecule, can give a fluorescent signal that could not have been detected in a protein complex because it was quenched, for example by a quencher placed on the protein.

Preferably, the method of the invention is characterized by the fact that in addition to the step in which the sample containing the RNA molecule pool with RNA binding protein is incubated to form RNA-RNA binding protein complexes, the method further comprises the steps in which:
a. an RNA binding protein is produced in bacterial protein overexpression systems,
b. the RNA binding protein is purified,
c. the RNA binding protein is prepared in a free form or, optionally, in a form bound to the reporter molecule,
d. the RNA binding protein is immobilized on the support before the step of incubation with RNA or, optionally, the RNA binding protein is immobilized on the support after the step of incubation with RNA in the form of RNA-RNA binding protein complexes,
e. after the sample containing the RNA molecule pool with the RNA binding protein is incubated, the unbound RNA molecules and optionally the released reporter molecule are washed away,
f. optionally, RNA is released from the RNA-RNA binding protein complexes,
g. selected and/or enriched RNA molecules are detected in the form of released RNA molecules or in the form of RNA-RNA binding protein complexes.

According to the invention, the RNA binding protein is preferably produced in a recombinant form in Escherichia coli bacteria or other suitable bacterial protein overexpression systems. The RNA binding protein purification is carried out by affinity chromatography and/or gel filtration or another suitable method. Preferably, a nickel support or other suitable bed is used to immobilize the RNA binding protein or RNA-RNA binding protein complexes. The release of RNA from the complexes is preferably carried out by proteinase K digestion or by phenol and chloroform extraction or another suitable method, with the release of RNA from the complexes being optional. There is no need to release RNA from complexes before detection; in such cases RNA is detected in the form of RNA-protein complexes. Detection of selected and/or enriched RNA molecules is preferably carried out by reverse transcription and amplification of nucleic acids, RT-PCR, or binding of the tagged probe to RNA, or by another suitable method, or, optionally is determined based on the presence of the released reporter molecule.

Any suitable method known in the art may be used to carry out each of the above steps of the method of the invention.

Preferably, the method of the invention is characterized in that the RNA molecules selected and/or enriched by this method are RNA molecules, especially mRNA, with a Cap 0 structure or a triphosphate group at the 5′ end, the Cap 0 structure comprises m7Gppp and/or its forms differing in methylation, such as Gppp. Furthermore, the method of the invention is characterized in that the selected and/or enriched RNA molecules are RNA molecules, especially mRNA, which contain a region of at least 4 unpaired nucleotides at the 5′ end. This is due to the fact that the binding of IFIT proteins to RNA is not dependent on the sequence but rather on the availability of the first few nucleotides. Due to RNA thermal denaturation, all the 5′ ends can be released from the secondary structure and become available to proteins.

Preferably, the method of the invention is also characterized by the fact that the selected and/or enriched RNA molecules are RNA molecules, especially mRNA, of viruses, bacteria, fungi, protozoa, invertebrates and/or plants, or vertebrate mtRNA, or RNA being an intermediate in the RNA maturation process. The method can be successfully used without species-based restrictions, but with a clear division into lower eukaryotic organisms and plants, whose mRNA contains Cap 0 recognized by the IFIT1 protein, and bacteria, whose mRNA contains a triphosphate group at the 5′ end recognized by the IFIT5 protein. IFIT1 specifically and selectively binds RNA to the Cap 0 structure at the 5′ end. IFIT5 specifically and selectively binds RNA to the ppp structure at the 5′ end, thereby recognizing bacterial mRNA.

Thus, the method of the invention enables the RNA selection and enrichment, especially with mRNA molecules, having the desired 5′ end characteristics of the RNA molecule, i.e. the RNA with a Cap 0 structure and/or a triphosphate group at the 5′ end, from the RNA molecule pool. The new RNA selection and enrichment method of the invention utilizes the properties of IFIT proteins which specifically bind to RNA molecules with suitably modified 5′ ends. It has been proved that the IFIT1 protein, placed in a mixture of synthetic RNA molecules with different 5′ ends (OH, P, ppp, Cap 0), specifically binds to RNA with the Cap 0 structure, while the IFIT5 protein specifically binds to RNA containing the triphosphate group (ppp) at the 5′ end.

Accordingly, the method of the invention utilizes the IFIT1 protein for enrichment of the RNA sample with molecules having the Cap 0 structure (m7Gppp or Gppp), which include mRNA, snRNA and other non-coding RNAs of lower eukaryotic organisms and plants, as well as IFIT5 protein for enrichment of the RNA sample with molecules having a triphosphate group (ppp) at the 5′ end, which include bacterial mRNA and some of sRNAs.

The RT-qPCR method also has shown the enrichment of certain RNAs from the total cellular RNA pool by means of human IFIT proteins: certain yeast mRNAs (having the Cap 0 end) were enriched by pulldown on IFIT1, and bacterial sRNAs and mRNAs (containing 5′-ppp)—by pulldown on IFIT5. Thus, the use of IFIT proteins may allow the isolation or enrichment of RNA molecules having the desired 5′-end characteristics. RNA molecules bound to IFIT1 and IFIT5 can be released from complexes with proteins and subjected to further examination, e.g. by electrophoretic separation, RT-qPCR or sequencing.

The invention furthermore relates to an RNA molecule selected and/or enriched by the method of the invention. Preferably, it is an RNA molecule, especially an mRNA, with a Cap 0 structure or a triphosphate group at the 5′ end, wherein the Cap 0 structure comprises m7Gppp and/or its forms differing in methylation, such as Gppp. Preferably, the RNA molecule of the invention has a region of at least 4 unpaired nucleotides at the 5′ end, making it accessible to IFIT proteins.

Preferably, the RNA molecule selected and/or enriched by the method of the invention is an RNA molecule, especially mRNA, of viruses, bacteria, fungi, protozoa, invertebrates and/or plants, or vertebrate mtRNA, or RNA being an intermediate in the RNA maturation process, with a clear division into lower eukaryotic organisms and plants, whose mRNA will be bound by the IFIT1 protein, whereas the bacterial mRNA having a triphosphate group at the 5′ end will be bound by the IFIT5 protein.

The subject of the invention is also the use of an RNA molecule selected and/or enriched by the method of the invention for detection in RNA pathogen-based diagnostic tests. Preferably, in this use the pathogen RNA is RNA of viruses, bacteria, fungi, protozoa and/or invertebrates, and the pathogen RNA molecule detection is carried out by reverse transcription and amplification of nucleic acids, RT-PCR, or binding of a tagged probe to pathogen RNA, or another suitable method, or optionally determined based on the presence of the released reporter molecule.

Accordingly, the method of the invention can be used to enrich pathogen RNA in samples taken from patients suspected of being infected to detect the presence of bacteria, viruses, yeast or parasites. Enrichment of pathogen RNA in the test sample, prior to using RNA detection methods, significantly increases the sensitivity and selectivity of RNA pathogen-based diagnostic tests.

The invention furthermore relates to the use of an RNA molecule selected and/or enriched by the method of the invention for preparation cDNA libraries for RNA sequencing or for preparing samples for direct RNA sequencing, and preferably for high-throughput RNA sequencing, in particular NGS sequencing.

The method for RNA selection and enrichment according to the invention enables to prepare cDNA libraries for sequencing of the new generation of RNA-Seq, based on a RNA pool enriched with mRNA molecules (having a suitably modified 5′ end, i.e. ppp e.g. in bacteria and Cap 0 e.g. in yeast) and thus allows to remove from the examined sample the RNA molecules having other (not recognized by the IFIT protein) groups at the 5′ ends, including ribosomal and transporting RNA (rRNA and tRNA), which usually hinder the RNA-Seq procedure. Thus, the method of the invention can be successfully used to enrich RNA-Seq libraries in experiments aimed at, among others, analysis of the transcriptome fraction, which is responsible for coding proteins. NGS libraries prepared by the method of the invention are significantly enriched, first of all in protein coding sequences, but also in molecule sequences that have appropriately modified 5′ ends and are usually found in cells in negligible amounts, e.g. regulatory RNA.

It is also possible to find additional applications for IFIT proteins in other molecular biology procedures, for example for identifying group identity at the 5′ end of RNA in organisms less known in terms of RNA metabolism. Thus, using IFIT proteins in the method of RNA sample selection and enrichment will have additionally cognitive significance due to the fact that the status of the 5′ ends for some RNAs (e.g. regulatory) has not yet been determined. In such cases, enrichment of NGS libraries with given sequences will also constitute the information about the modification of their end 5′.

The method for RNA sample selection and/or enrichment of the invention is universal, fast, based on simple laboratory techniques, and also economically advantageous.

The method of the invention is universal and reproducible for most lower eukaryotic organisms, all bacteria and some viruses. The method can be successfully used without any species-based restrictions, but with a clear division into lower eukaryotic organisms and plants, whose mRNA contains Cap 0 recognized by the IFIT1 protein, and bacteria, whose mRNA contains a triphosphate group at the 5′ end recognized by the IFIT5 protein. IFIT1 specifically and selectively binds to RNA having the Cap 0 structure at the 5′ end. It distinguishes 2′O methylation on the first nucleotides, i.e. distinguishes Cap 0 from Cap 1 and Cap 2, and owing to that it recognizes mRNAs of lower eukaryotes and plants. IFIT5 specifically and selectively binds to RNA having the ppp structure at the 5′ end, thereby recognizing bacterial mRNA. IFIT proteins do not bind molecules whose 5′ end contains a monophosphate group (e.g. rRNA, tRNA) or —OH (RNA breakdown products, e.g. products of RNA autocatalysis or preparation-associated breakdown during isolation). The selection carried out using them will be independent of the RNA nucleotide sequence. In addition, due to the fact that Cap 0 is added to mRNA molecules co-transcriptionally and primarily during the RNA maturation process, the pool of IFIT1-bound molecules should contain both mature mRNA and messenger RNA precursor molecules (pre-mRNA) at various stages of splicing. Thus, the method of the invention can be successfully used for species that are not model and for which dedicated commercial rRNA removal kits cannot be used. The proposed solution can be used interchangeably or together with other methods of enrichment an RNA sample with mRNA.

Furthermore, the method according to the invention is a fast and economically advantageous one, and its implementation is relatively simple. Selection and enrichment of the RNA sample lasts up to a few hours and requires no specialized laboratory equipment, utilizes simple laboratory techniques and devices that are standard equipment in most laboratories. Importantly, the procedure does not introduce RNA modification. The fact that IFIT proteins occur in cells under normal conditions, but only in vertebrates and under conditions induced by viral infection or inflammation induction, may give an additional advantage over other methods in the form of lower risk of artifacts resulting from the interaction of cellular proteins with the IFIT protein. In addition, the advantage of the IFIT proteins is easy, fast and efficient overproduction in bacterial protein expression systems. Tags are placed at the N-termini of the IFIT proteins that can be used to tag or immobilize the IFIT proteins on supports and do not affect their activity. The IFIT proteins are relatively small (IFIT1 56 kDa, IFIT5 58 kDa), stable (can be safely stored at −20° C. or −80° C. in the long term and at 4° C. in the short term) and susceptible to engineering. They do not show enzymatic activity associated with e.g. RNA modification or fragmentation, which means that the RNA bound to them can easily be recovered and used in subsequent procedures, for example for amplification and sequencing. They also do not require labile cofactors such as ATP.

The invention is illustrated in a drawing, in which:

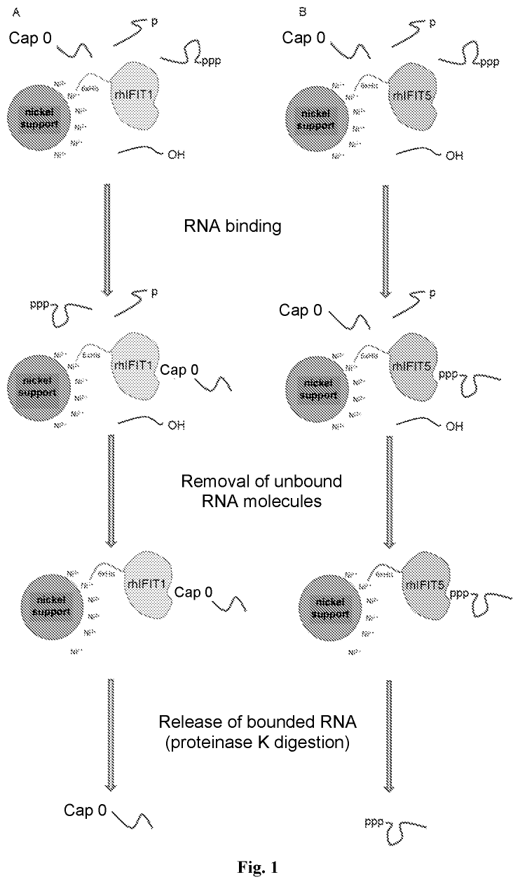

FIG. 1 is a scheme of the method of the invention illustrating the selection and enrichment of RNA molecules having appropriate modification of the 5′ end using the rhIFIT1 (A) and rhIFIT5 (B) proteins. Proteins are immobilized on a nickel support, and placed in a mixture of RNA molecules with different 5′ ends. After washing away the unbound RNA molecules, the rhIFIT1 complexes with 5′Cap 0-RNA and rhIFIT5 with 5′ppp-RNA are broken down and the RNA molecules released from the complexes are purified and analyzed.

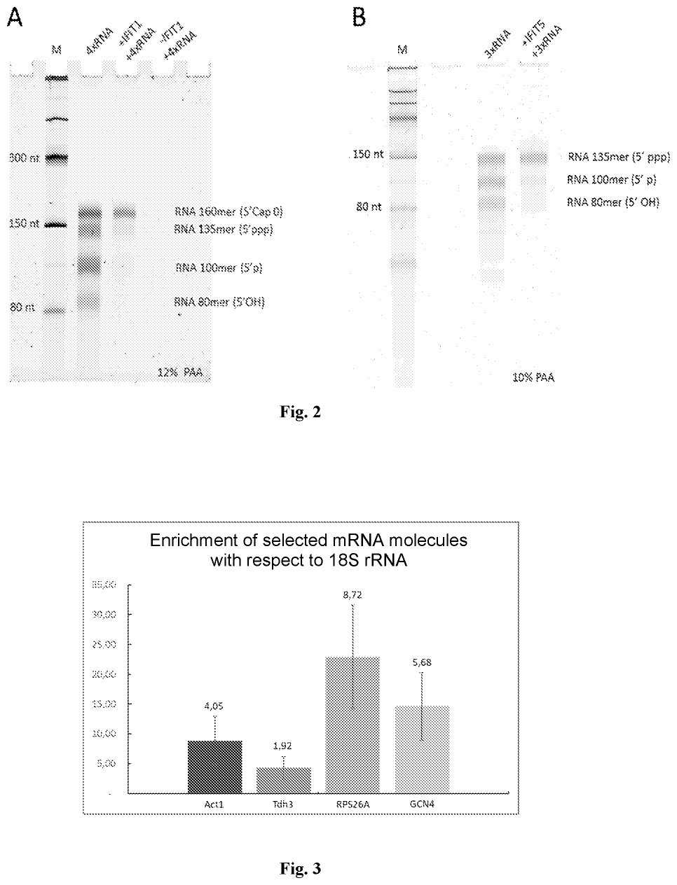

FIG. 2 shows images after RNA separation in a polyacrylamide gel of a mixture of synthetic RNA molecules with different 5′ ends and RNA molecules released from the complexes with IFIT1 (A) or IFIT5 (B) proteins following a pull-down experiment (test sample “+IFIT+RNA” and negative control “−IFIT+RNA”). M—size marker, PAA—polyacrylamide gel with a given polyacrylamide content.

FIG. 3 shows plots of enrichment of selected mRNA from Saccharomyces cerevisiae yeast following a pull-down reaction with rhIFIT1 with respect to 18S rRNA. RT-qPCR results were analyzed by relative quantification, Act1—actin 1, Tdh3—3-phosphoglycerin aldehyde dehydrogenase, RPS26A—component of the small ribosomal 40S unit, GCN4—general control protein 4.

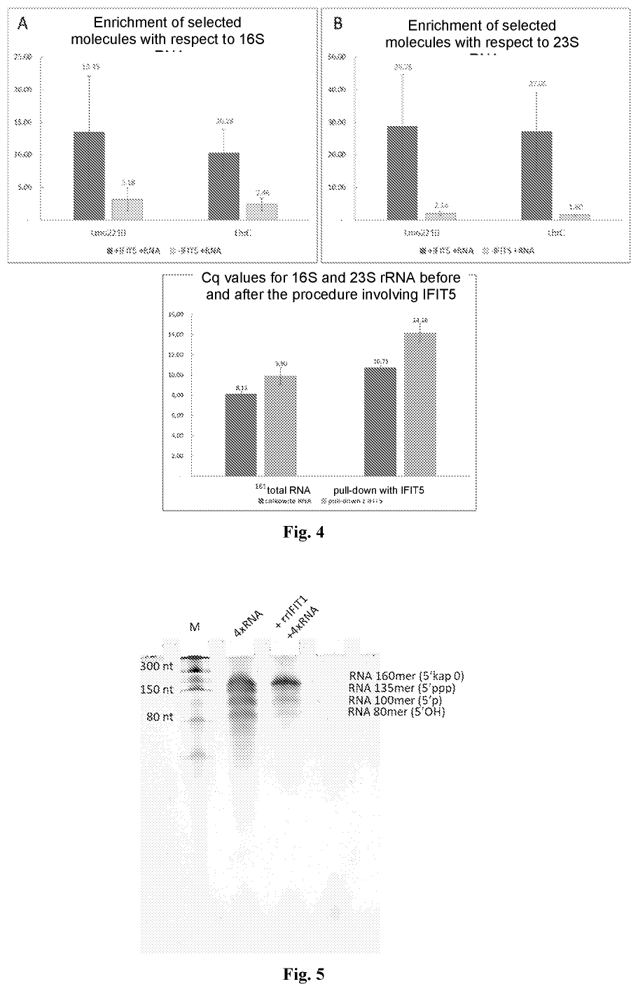

FIG. 4 shows plots of enrichment of selected RNA from Listeria monocytogenes bacteria following a pull-down reaction with IFIT5: A) with respect to 16S rRNA; B) with respect to 23S rRNA; C) an increase in Cq value means lower amounts of rRNA in samples subjected to pulldown with IFIT5 (e.g. a Cq difference of 1 means a two-fold decrease in RNA amount).

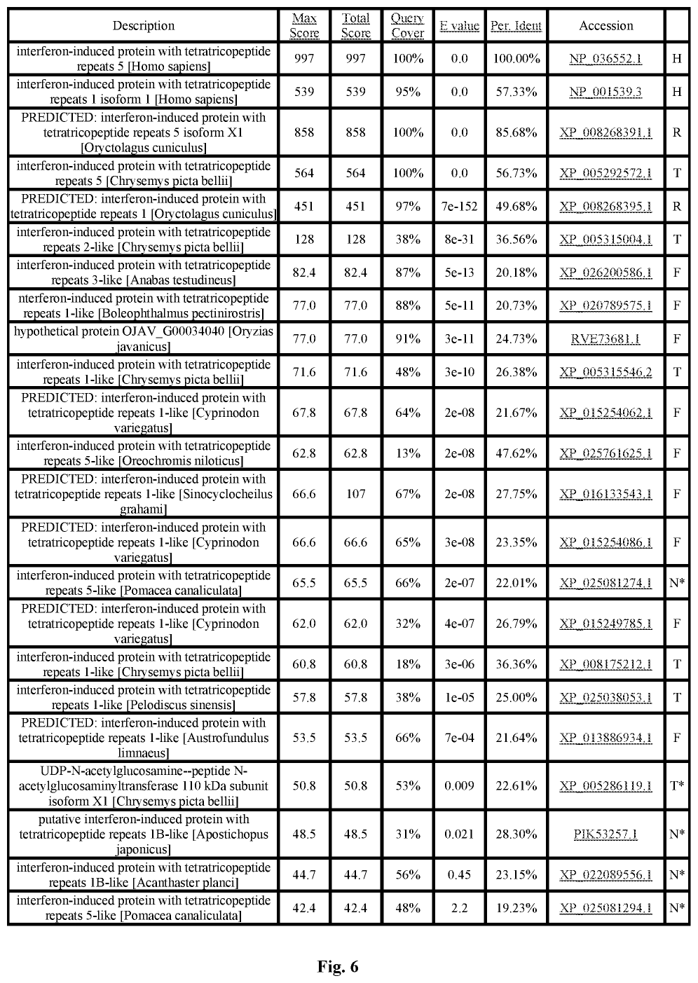

FIG. 5 shows the image after RNA separation in a polyacrylamide gel of a mixture of synthetic RNA molecules with different 5′ ends and RNA molecules released from rrIFIT1 protein complexes following a pull-down experiment (test sample+IFIT+RNA), M—size marker.

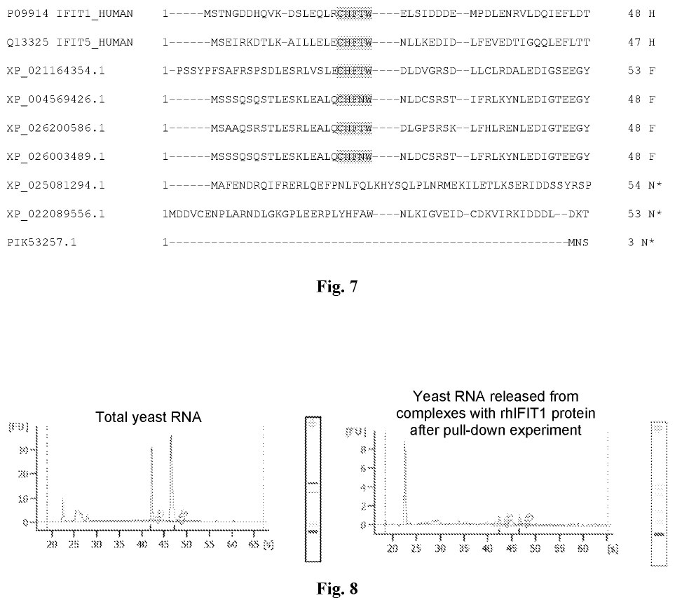

FIG. 6 shows a comparison and determination of the % of amino acid sequence identity of IFIT5 protein homologs in various animal species. Results of the search in the NCBI protein sequence database using the BLAST-P algorithm by means of the hIFIT5 sequence (SEQ ID NO: 2) have been collated. Parameters are given for selected sequences of human (H), rabbit (R), turtle (T), fish (F) and invertebrate (N) homologues. The proteins marked (*) in the last column are most likely TPR repeat proteins that are not structural and functional IFIT homologs because they do not contain the CHFxW motif (FIG. 7).

FIG. 7 shows the alignment of selected IFIT protein homologs in the region of the conserved CHFTW structural motif. H—human protein sequences, F—fish protein sequences, N*—invertebrate protein sequences most likely not being structural and functional counterparts of IFIT proteins.

FIG. 8 shows images of the separation during capillary electrophoresis in a Bioanalyzer, Agilent of RNA molecules obtained following the pull-down experiments described in Example 2 (A) and Example 3 (B).

The invention has been illustrated in detail in the following examples. The exemplary embodiments do not limit the scope of the invention.

### WORKING EXAMPLES

### Example 1

A Method for RNA Selection and/or Enrichment with RNA Molecules Having a Cap 0 Structure at the 5′ End and with RNA Molecules Having a Triphosphate Group at the 5′ End, from a Pool of Synthetic RNA Molecules with Different 5′ Ends

In the RNA selection and enrichment method, recombinant human protein IFIT1 (rhIFIT1) with SEQ ID NO:1 and recombinant human protein IFIT5 (rhIFIT5) with SEQ ID NO:2 sequence, both in the fusion form with a histidine tag (His-tag) were used.

The rhIFIT1 and rhIFIT5 proteins were obtained in a bacterial protein overexpression system using standard techniques, utilizing Escherichia coli BL21-CodonPlus (DE3)-RIL strain. Plasmid with rhIFIT1 or rhIFIT5 protein encoding sequence was introduced into bacterial cells by a heat shock method, and the bacterial clones that collected the introduced genetic material were selected by culturing on LB selection supports with antibiotics. The production of proteins was induced after the bacterial culture reached OD₆₀₀ 0.6 by adding isopropyl-β-D-galactopyranoside (IPTG) to the final concentration of 0.5 mM. After inducing the protein production, bacterial culture was continued overnight (about 16 hours) at 25° C., with shaking at 1200-1600 rpm. Once the culturing was ended, the bacteria were centrifuged (4000 rpm, 20 min. 4° C.), the supernatant was removed and the remaining sediment was suspended in a lysis buffer (50 mM Tris pH 7.5, 0.5 M NaCl, 20 mM imidazole, 10% glycerol, 0.5 mM TCEP), to which protease inhibitor and lysozyme (Sigma) were added. The bacterial suspension was sonicated (breaking down by ultrasounds), then centrifuged (20000 rpm, 40 min. 4° C.) and a clear bacterial lysate was collected containing, among others, IFIT soluble proteins produced in bacterial cells.

The IFIT proteins were purified by affinity chromatography and gel filtration. For this purpose, the bacterial lysate was passed through a HisTrap HP crude column (GE Healthcare Life Sciences). The rhIFIT1 or rhIFIT5 protein was eluted from the column in a buffer gradient with a high content of imidazole (50 mM Tris pH 7.5, 0.5 M NaCl, 500 mM imidazole, 10% glycerol, 0.5 mM TCEP). The collected fractions containing the purified protein were combined. The sample was diluted 5× in a buffer containing 50 mM Tris pH 7.5, 10% glycerol, 0.5 mM TCEP and passed through a HiTrap Heparin HP column (GE Healthcare Life Sciences) equilibrated with a heparinA buffer (50 mM Tris pH 7.5, 100 mM NaCl, 10% glycerol, 0.5 mM TCEP). The rhIFIT1 or rhIFIT5 protein was eluted from the heparin column in a heparinB buffer gradient with a high salt content (50 mM Tris pH 7.5, 1 M NaCl, 10% glycerol, 0.5 mM TCEP). The collected fractions containing the purified protein were combined. The sample was concentrated to a volume of 1 ml using membrane filters (Vivaspin 20, Sartorius) and passed through a Superdex 200 Increase column (GE Healthcare Life Sciences) in a SEC buffer (50 mM Tris pH 7.5, 150 mM NaCl, 5% glycerol, 0.5 mM TCEP). The rhIFIT proteins purified in this manner were stored at 4° C. for a few days or frozen in a stream of liquid nitrogen and stored at −80° C.

The rhIFIT1 and rhIFIT5 proteins were then used for pull-down experiments, which were started by immobilizing the protein to a Ni Sepharose 6 Fast Flow nickel resin support (GE Healthcare). For this purpose, 10 μl of the support was transferred to an Eppendorf tube, washed with 1 ml of RNase-free water, followed by 1 ml of Binding Buffer (BB) of the following composition: 50 mM Tris pH 7.5, 150 mM NaCl, 1 mM DTT, 5 mM imidazole, 3 mM MgCl₂, 0.01% Tween 20, centrifuging each time at 100 rpm, 4° C., for 1 min. and removing supernatant. 1 ml of the BB buffer and 2 μg (35 pmol) of rhIFIT1 or rhIFIT5 protein were added to the prepared support. The sample was incubated for 30 min. at 4° C. with gentle mixing. Once the protein was bound to the support, the sample was centrifuged as before, the support was washed with 1 ml of the BB buffer and centrifuged again.

1 ml of a mixture containing the BB buffer, about 10 μg of RNA and poly (dIdC) (Poly (deoxyinosinic-deoxycytidylic) acid sodium salt, Sigma) was added to the support at a final concentration of 2 μg/ml. Protein binding to RNA was carried out for 60 min. at 4° C. with gentle mixing. Alternatively, protein binding to RNA was performed prior to immobilizing the protein on the support, starting by mixing 2 μg rhIFIT1 or rhIFIT5 protein with about 10 μg of RNA in 1 ml of the BB buffer. Protein binding to RNA was carried out for 60 min. at 4° C. with gentle mixing. After that the formed protein complexes with RNA were immobilized on a nickel support by incubating the sample with an addition of poly(dIdC) at a final concentration of 2 μg/ml with the support (washed with water and the BB buffer) for 30 min. at 4° C. with gentle mixing. In both cases, after incubation and immobilization, RNA molecules that did not bind to the protein were removed by washing them away. For this purpose, RNA-protein complexes immobilized on the nickel support were washed three times with 1 ml of a Wash Buffer (WB) of the composition: 50 mM Tris pH 7.5, 250 mM NaCl, 1 mM DTT, 5 mM imidazole, 3 mM MgCl₂, 0.01% Tween 20, each time being centrifuged at 100 rpm, 4° C., for 1 min., followed by supernatant removal.

In the last step, RNA was released from the complexes with IFIT1 and IFIT5, by removing proteins. For this purpose, 400 μL of the WB buffer with proteinase K (Sigma) was added to the support with immobilized complexes at a final concentration of 50 μg/ml. The sample was incubated for 60 min. at 37° C., with shaking at 800 rpm, then centrifuged at 100 rpm, 4° C., for 1 min. The supernatant was collected, 5 μl of linear acrylamide (Sigma) and 2.5 volumes of 100% ethanol were added to it. Alternatively, 400 μL of the WB buffer was added to the support with immobilized complexes and then RNA was purified by phenol and chloroform extraction. RNA was precipitated by adding 1/10 volume of 3M sodium acetate pH 4.8, 5 μl of linear acrylamide (Sigma) and 2.5 volumes of 100% ethanol to the purified sample. The samples were incubated overnight at −20° C., then centrifuged for at least 30 min. at 4° C. at maximum speed. The supernatant was removed and the pellet was washed with 1 ml 80% ethanol and centrifuged for 15 min as before. The RNA precipitate was dried (5-10 minutes) at room temperature, then dissolved in 10-20 μL RNase-free water.

A scheme illustrating the described pull-down experiment aimed for the selection and enrichment of RNA molecules by means of IFIT proteins is shown in FIG. 1.

A pull-down using rhIFIT1 or rhIFIT5 protein and a pool of synthetic RNA molecules with different 5′ end was carried out to check whether it is possible to select and/or enrich the RNA sample with RNA molecules having a Cap 0 structure at the 5′ end and with RNA molecules having a triphosphate group at the 5′ end by means of rhIFIT proteins. For this purpose, a mixture was prepared of four types of synthetic RNA molecules, obtained by in vitro transcription (using the HiScribe™ T7 In Vitro Transcription Kit, New England BioLabs, according to the manufacturer's protocol) and enzymatic modification of 5′ ends. The 5′OH modification was obtained by incubating RNA with CIP dephosphorylase (Alkaline Phosphatase, Calf Intestinal, New England BioLabs, according to the manufacturer's protocol). Modification of 5′p was obtained by RNA digestion with 5′ RNA pyrophosphohydrolase (RppH, New England BioLabs, according to the manufacturer's protocol). The 5′ppp modification is a natural effect of in vitro transcription. The 5′ Cap 0 modification was achieved using the capping enzyme of Vaccinia virus (using the Vaccinia Capping System kit, New England BioLabs, according to the manufacturer's protocol). The mixture was prepared by mixing 70 picomoles of each type of molecules:

RNA 80mer (5′OH) 2 μg (70 pmol)

RNA 100mer (5′p) 2.4 μg (70 pmol)

RNA 135mer (5′ppp) 3.2 μg (70 pmol)

RNA 160mer (5′Cap 0) 3.8 μg (70 pmol)

The RNA sample prepared in this way was added to the rhIFIT1 or rhIFIT5 protein immobilized on the nickel support and the protein was bound to RNA followed by recovery of the selected RNA molecules from the complexes following the pull-down protocol described above. The obtained RNA was separated by electrophoretic separation in a 12% polyacrylamide gel containing 7M urea, and finally visualized by staining the gel with SYBR™ Gold Nucleic Acid Gel Stain (Thermo Fisher Scientific) and documented using a ChemiDoc MP Imaging System Bio-Rad (FIG. 2).

The results presented in FIG. 2 show that significant enrichment of the sample with RNA molecules having a Cap 0 structure compared to the remaining RNAs was obtained when using the rhIFIT1 protein and in RNA molecules with a triphosphate group when using the rhIFIT5 protein. RNA samples recovered from the complexes with rhIFIT1 and rhIFIT5 proteins are of good quality (no signs of RNA degradation), which allows to use them in further procedures: transcription into DNA, amplification and sequencing or detection with a tagged probe. Therefore, an efficient, fast and simple method of selecting and enriching the tested RNA sample with RNA molecules having a Cap 0 structure and/or a triphosphate group at the 5′ end was developed.

On this basis, it was assumed that the RNA selection and enrichment method using the rhIFIT1 and rhIFIT5 proteins of the invention is universal and allows to selectively capture RNA molecules with Cap 0 and a triphosphate group at the 5′ end also in complex tests of total RNA isolated from cells or tissues of various organisms whose RNA has specific Cap 0 and ppp structures at the 5′ end.

To verify the above, pull-down experiments analogous to those described above were performed using rhIFIT1 protein and total RNA isolated from Saccharomyces cerevisiae yeast, strain BY4741 (Example 2), as well as for rhIFIT5 protein with total RNA from Listeria monocytogenes bacteria, strain EGDe (Example 3) cultured under stress conditions stimulating transcription of active genes during infection. On this basis, the versatility of the method in terms of the origin of the RNA sample was concluded, since the method allows to enrich RNA molecules from organisms belonging even to distant kingdoms such as fungi or bacteria.

In addition, in order to confirm that in the method of the invention not only human-derived rhIFIT1 and rhIFIT5 proteins can be used, but also non-human homologues of the rhIFIT1 and rhIFIT5 proteins, the amino acid sequence of these proteins was compared (Example 5) and in further experiments a rabbit homolog of rhIFIT1 protein (Example 4) and fish homolog of rhIFIT5 protein (Example 4) were used.

### Example 2

Method for Selection and/or Enrichment of RNA Isolated from Yeast with mRNA Molecules Having a Cap 0 Structure at the 5′ End Using the rhIFIT1 Protein

The selection and enrichment method, including pull-down experiments, was carried out analogous to that described in the Example 1, with the difference that the RNA molecule pool was a total RNA sample isolated from Saccharomyces cerevisiae yeast, strain BY4741, by extraction using phenol and chloroform. The protein used was rhIFIT1 with the sequence SEQ ID NO: 1, fused to the His-tag.

After pull-down type experiments, the RNA released from the complexes with the rhIFIT1 protein was subjected to a reverse transcription reaction (using a commercial kit, e.g. First Strand cDNA Synthesis Kit, Roche, according to the manufacturer's protocol). The resulting cDNA was used for real-time quantitative PCR (RT-qPCR). The results were analyzed by relative quantification, comparing the level of selected transcripts: Act1 (actin 1), Tdh3 (3-phosphoglycerylaldehyde dehydrogenase), RPS26A—(component of the small ribosomal 40S unit), GCN4 (general control protein 4, transcription factor) to the level of 18S rRNA. The enrichment of the material with selected RNA molecules was then determined by comparing the relative amount before and after the pull-down procedure.

The results presented in FIG. 3 show that the use of the rhIFIT1 protein enriches the yeast-derived RNA sample with mRNA molecules from a few to several dozen times relative to 18S rRNA.

### Example 3

A Method for Selection and/or Enrichment of RNA Isolated from Bacteria with RNA Molecules Having a Triphosphate Group at the 5′ End Using the rhIFIT5 Protein

The selection and enrichment method, including pull-down experiments, was carried out analogous to that described above in the Example 1, with the difference that the RNA molecule pool was a total RNA sample isolated from Listeria monocytogenes bacteria cultured under stress conditions stimulating transcription of virulence-associated genes and the protein used was rhIFIT5 with the sequence SEQ ID NO: 2, fused to a His-tag.

RNA released from the complexes with the rhIFIT5 protein after pull-down experiments was subjected to reverse transcription (using a commercial kit, e.g., the First Strand cDNA Synthesis Kit from Roche). The resulting cDNA was used for real-time quantitative PCR (RT-qPCR). The results were analyzed by relative quantification, comparing the level of selected transcripts to the level of ribosomal RNA. The enrichment of the material with selected RNA molecules was then determined by comparing the relative amount before and after the pull-down procedure.

The results are shown in FIG. 4. Following the procedure using the rhIFIT5 protein, approximately 10 fold enrichment relative to 16S rRNA (FIG. 4A) and over 20 fold enrichment relative to 23S rRNA (FIG. 4B) was noted. Enrichment with molecules belonging to the mRNA fraction (Lmo2210) as well as with small non-coding sRNA (LhrC) took place.

### Example 4

A Method for Selection and/or Enrichment of RNA with RNA Molecules Having a Cap 0 Structure at the 5′ End Using an IFIT Protein Homologue from a Non-Human Organism—Example of rrIFIT1 (Rabbit Homologue of rhIFIT1 Protein) or rfIFIT12B (Fish Homolog of IFIT1 Protein)

The selection and enrichment method, including pull-down experiments, was carried out analogous to that described above in the Example 1, with the difference that the protein was a rabbit protein rrIFIT1 with the sequence SEQ ID NO: 3 or a fish protein rfIFIT12B with the sequence SEQ ID NO: 4, fused with SUMO-tag and His-tag. A mixture of four types of synthetic RNA molecules with different 5′ ends obtained by in vitro transcription was prepared as described in Example 1.

The results presented in FIG. 5 show that, as in the case of rhIFIT1, a significant enrichment of the sample with RNA molecules having a Cap 0 structure was obtained with the rabbit homolog. RNA samples recovered from the protein complexes are of good quality (no signs of RNA degradation), they can be used in further procedures: transcription into DNA, amplification and sequencing or detection with a tagged probe.

On this basis, it has been concluded that the RNA selection and enrichment method utilizing the IFIT1 protein homologue from a non-human organism allows selective capture of RNA molecules with Cap 0.

### Example 5

### Comparison and Determination of Amino Acid Sequence Conservation of IFIT1 and IFIT5 Protein Homologs in Various Animal Species

The amino acid sequence comparison was performed using the BLASTp algorithm, searching the NCBI database, available at https://blast.ncbi.nlm.nih.gov, using the human IFIT1 (SEQ ID: 1) or IFIT5 (SEQ ID: 2) protein sequences. The results obtained for the selected sequences found are shown in FIG. 6, where the scores and % of sequence identity are given for a given human IFIT5 sequence coverage in individual alignments. Examples of homologous IFIT proteins from various organisms were selected—human, rabbit, turtle and fish, with some fish IFIT homologs having a sequence that was similar in approximately 20% to the human IFIT5 protein sequence. IFIT1 and IFIT5 protein homologs were found to be widespread among vertebrates and the lower limit of sequence identity for probable IFIT homologs was set to 20% for the coverage of most sequences of a human IFIT protein.

In FIG. 6 also several examples of invertebrate proteins were provided that were designated in the NCBI database as IFIT protein homologs, as well as the UDP-N-acetylglucosamine-peptide N-acetylglucosaminyltransferase enzyme, which was a frequent search result. Some of these proteins have TPR motifs and % of sequence identity exceeding 20% with respect to human IFIT proteins, although they are unlikely structural and functional equivalents of the IFIT protein. Therefore, an auxiliary criterion for identifying IFIT protein homologues was introduced, which is the presence of a structurally important motif having the sequence CHFxW (where x is an amino acid that is usually T). The CHFxW motif adopts a helical twist conformation between alpha-helices in the subdomain I of IFIT proteins and is most likely important for the correct folding or stability of the IFIT protein. Structural homologues of the IFIT proteins, for which the full sequence is available in the NCBI database, are characterized by the presence of the CHFxW motif, as shown by the alignment of IFIT proteins in this region presented in FIG. 7 (achieved using the Clustal Omega algorithm available in NCBI tools).

### Example 6

Evaluation of the Quality and Composition of the RNA Sample Obtained by the Method of the Invention after Pull-Down Experiments

RNA molecules selected by the method of the invention described in the Example 1 using rhIFIT1 and total RNA from yeast as well as rhIFIT5 and total RNA from bacteria were analyzed by capillary electrophoresis in a Bioanalyzer (with Agilent RNA 6000 Nano Kit according to the manufacturer's protocol).

It has been shown that the rRNA fraction in RNA samples obtained by pull-down experiments is significantly reduced (FIG. 8), suggesting that the method of the invention can be used for ribodepletion (removal of rRNA) and enrichment with mRNA molecules and some non-coding regulatory RNAs to prepare for a sample for RNA sequencing.

### Example 7

### Enrichment of RNA Material by the Method of the Invention for the Preparation of an RNA Sample for High-Throughput Nanopore-Based Sequencing

RNA molecules selected by the method of the invention described in the Example 1 using rhIFIT1 and total RNA from yeast as well as rhIFIT5 and total RNA from bacteria were used to prepare RNA for direct nanopore sequencing. RNA released from complexes with the IFIT proteins were polyadenylated (Poly(A) (using the Tailing Kit, Invitrogen according to the manufacturer's protocol). Then high-throughput sequencing was performed using the MinION device (Oxford Nanopore, according to the manufacturer's protocol).

Preliminary analysis of the obtained results showed that the RNA sample after pull-down experiments was enriched with mRNA molecules, while the rRNA and tRNA fraction decreased.

### Example 8

### Enrichment of RNA Material by the Method of the Invention for Detecting Pathogen RNA in Diagnostic Tests

The method for selection and/or enrichment of RNA according to the invention, including pull-down experiments, was carried out analogous to that described in the Example 3. Then, the enriched RNA was further analyzed for the presence of specific RNA derived from the Listeria monocytogenes pathogen in the sample tested by a colorimetric test using a commercial RT-LAMP kit (e.g. WarmStart® Colorimetric LAMP 2X Master Mix (DNA & RNA), New England Biolabs, as per manufacturer's recommendations). The RT-LAMP assay combines reverse transcription, isothermal nucleic acid amplification and reading by changing the color of the reaction.

Preliminary results showed enrichment of the RNA obtained by the method of the invention compared to the initial sample as well as increased test sensitivity resulting from the method. It has been shown that the enrichment of RNA molecules can be a diagnostic test step for detecting the pathogen presence. Enrichment of samples taken from patients with suspected infection with pathogen RNA material increases the sensitivity and selectivity of diagnostic tests.

Sequence listing
amino acid sequence of human protein IFIT1
SEQ ID NO: 1
MSTNGDDHQVKDSLEQLRCHFTWELSIDDDEMPDLENRVLDQIEFLDTKYS
VGIHNLLAYVKHLKGQNEEALKSLKEAENLMQEEHDNQANVRSLVTWGNFA
WMYYHMGRLAEAQTYLDKVENICKKLSNPFRYRMECPEIDCEEGWALLKCG
GKNYERAKACFEKVLEVDPENPESSAGYAISAYRLDGFKLATKNHKPFSLL
PLRQAVRLNPDNGYIKVLLALKLQDEGQEAEGEKYIEEALANMSSQTYVFR
YAAKFYRRKGSVDKALELLKKALQETPTSVLLHHQIGLCYKAQMIQIKEAT
KGQPRGQNREKLDKMIRSAIFHFESAVEKKPTFEVAHLDLARMYIEAGNHR
KAEENFQKLLCMKPVVEETMQDIHFHYGRFQEFQKKSDVNAIIHYLKAIKI
EQASLTRDKSINSLKKLVLRKLRRKALDLESLSLLGFVYKLEGNMNEALEY
YERALRLAADFENSVRQGP
amino acid sequence of human protein IFIT5
SEQ ID NO: 2
MSEIRKDTLKAILLELECHFTWNLLKEDIDLFEVEDTIGQQLEFLTTKSRL
ALYNLLAYVKHLKGQNKDALECLEQAEEIIQQEHSDKEEVRSLVTWGNYAW
VYYHMDQLEEAQKYTGKIGNVCKKLSSPSNYKLECPETDCEKGWALLKFGG
KYYQKAKAAFEKALEVEPDNPEFNIGYAITVYRLDDSDREGSVKSFSLGPL
RKAVTLNPDNSYIKVFLALKLQDVHAEAEGEKYIEEILDQISSQPYVLRYA
AKFYRRKNSWNKALELLKKALEVTPTSSFLHHQMGLCYRAQMIQIKKATHN
RPKGKDKLKVDELISSAIFHFKAAMERDSMFAFAYTDLANMYAEGGQYSNA
EDIFRKALRLENITDDHKHQIHYHYGRFQEFHRKSENTAIHHYLEALKVKD
RSPLRTKLTSALKKLSTKRLCHNALDVQSLSALGFVYKLEGEKRQAAEYYE
KAQKIDPENAEFLTALCELRLSI
amino acid sequence of rabbit protein IFIT1
SEQ ID NO: 3
MWNPQRTRARSQRAQSGSGNLQTSASFSATMSECAEEHPLKDRLQKLRCHF
TWGLLIEDTGLPDLEDRILEEIQFLDTENKVGYNLLAYVKHLQGKHEDALE
NLKEAEEVVQGDQADHSDVRSLVTWGNYAWVHYHMGRLADAQTYLDKVENT
CQKSADPTRYSTQCPEMDCEEGWALLKCGGKNYERAKACFEKALEADPENP
EFNTGYAITVYRLDYPAKRPCDVSDAFSLQPLRKAIRLNPQDAYLKALLAL
KLQDVGEEAEGRECLEEALAHTSSQTYVFRYAAKFFRRQGRVDEALKYLKM
ALKATPSSAFLHQQIGLCYKKKTNQIMNATHMQPTRQDRENVDRLIQLAIF
HFEYAVKQKPTFEVAYVDLARMYITAGDHEKAEDTFQKVLCMTPLQEHIQQ
NIHFSYGQFQQFQKKSEVDAITHYLQAVTIRKDSYARDKSIKALEQLVSWK
LERNPLDQEALSLREVLHRLVGGRDEALECSEQDLRLAADSGNWVGSSL
amino acid sequence of fish protein IFIT12B of
Danio rerio
SEQ ID NO: 4
MTSDVSSMEADRALRTKLHQLECHFTWALIKDDIDINDLLNRLEEQINLDL
EKKERLARTYSALAYVQYLLGFHEKAHQSLMTSKKLHIESHGDEFYRTLIV
TYGNLAWLNYHMKNYTECESYLNSLQRINETSPAEFSSIPEVLGEKGWTFL
KFSRKYYDGAKECFRKAVELEPEEPEWHTGYAIALYRTEFESTVLEDSATV
KQLRLAIEMNPDDDVLKVLLSLRLIVYKRYGEAESWVEKALEKSPDHPHVM
RYVGKFFRNKGCVDRSIDLLKRALERSPNSSFIHHQLALCYKYKKIQVLQE
QSHHARGSRVQQLRDQCIFHLEKATSLTTSFISAMSDLALQYGENGDIPRA
EELFQVTFKIAKEKNDGLHVVNYYYAEYQLYCHRCEPLAVQHYMECLKMCP
KSVEGRISSTRMKKTAEKWIDRKSQEGKAYGMLAFLHKVKGEIAQAIECYE
KALSYEDNNEFLRNLRELRLSLL

1-31. (canceled)

32. A method for RNA selection and/or enrichment, especially in mRNA molecules, from a pool of RNA molecules, the method comprising the following steps:
(a) preparing an RNA binding protein
(b) binding a bed to RNA molecules by means of RNA binding protein
(c) washing away the unbound RNA molecules
(d) detecting selected RNA molecules
whereby a protein of the IFIT protein family or its functional variants, homologues or mutants are is used as the RNA binding protein.

33. The method according to claim 32, wherein the RNA binding protein for use in step (a) is used as a result of conducting the following steps:
(a1) producing the RNA binding protein in bacterial protein overexpression systems
(a2) purifying the RNA binding protein.

34. The method according to claim 32, wherein the RNA binding protein for use in step (a) is in a form bound to a reporter molecule.

35. The method of claim 34, wherein a released reporter molecule is also washed away in step (c).

36. The method according to claim 32, wherein the binding the bed to RNA by means of RNA binding protein in step (b) is achieved in the following steps:
(b1) immobilizing the RNA binding protein on the bed
(b2) incubating a sample comprising a pool of RNA molecules with the RNA binding protein to form RNA-RNA binding protein complexes.

37. The method of claim 32, wherein the binding the bed to RNA by means of RNA binding protein in step (b) is achieved in the following steps:
(b1′) incubating a sample comprising a pool of RNA molecules with the RNA binding protein to form RNA-RNA binding protein complexes
(b2′) immobilizing the RNA binding protein in form of RNA-RNA binding protein complexes on the bed.

38. The method according to claim 32, wherein after the step (c) and before the step (d) the RNA molecules are released from RNA-RNA binding protein complexes.

39. The method according to claim 32, wherein detecting of the RNA molecules in step (d) is performed based on the presence of the released reporter molecule

40. The method according to claim 32, wherein detecting the selected and detected RNA molecules are RNA molecules, especially mRNA, with a Cap 0 structure or a triphosphate group at the 5′ end

41. The method according to claim 40, wherein the Cap 0 structure comprises m7Gppp and/or its forms differing in methylation, such as Gppp.

42. The method according to claim 40, wherein the selected and detected RNA molecules are RNA molecules, especially mRNA, comprising a region of at least 4 unpaired nucleotides at the 5′ end.

43. A method according to claim 32, wherein the selected and detected RNA molecules are selected from:
mRNA molecules of viruses, bacteria, fungi, protozoa, invertebrates and/or plants; mtRNA molecules of vertebrates;
or RNA molecules being an intermediate in the RNA maturation process.

44. The method according to claim 32, wherein the protein of the IFIT protein family is a protein containing a tetratricopeptide repeat (TPR) region and a structural motif with an amino acid sequence CHFxW present in a helical twist in a loop between alpha-helices, where x means T or N or another amino acid.

45. The method according to claim 32, wherein the protein of the IFIT protein family is a protein comprising an amino acid sequence which selected from
(i) a sequence at least 20% identical to the amino acid sequence of the human IFIT1 protein shown as SEQ ID NO: 1, or a functional fragment thereof, and
(ii) a sequence at least 20% identical to the amino acid sequence of the human IFIT5 protein shown as SEQ ID NO: 2, or a functional fragment thereof.

46. The method according to claim 32, wherein protein of the IFIT protein family is selected from:
human IFIT1 protein having the amino acid sequence shown as SEQ ID NO: 1;
human IFIT5 protein having the amino acid sequence shown as SEQ ID NO: 2;
a functional variant of IFIT1 and/or IFIT5 protein from a non-human organism;
or mutants thereof.

47. The method according to claim 32, wherein the IFIT1 and/or IFIT5 protein is a recombinant protein.

48. The method according to claim 47, wherein the recombinant IFIT1 and/or IFIT5 protein additionally contains an oligohistidine tag, a Strep-tag, a One-Strep or other elements for purifying or immobilizing the protein, and optionally a protease cleavage site between the tag and the tag removal protein and optionally the SUMO tag or other domains improving the protein solubility or stability.

49. An RNA molecule selected and detected by the method defined in claim 32.

50. Use of the RNA molecule selected and detected by the method of claim 32 in RNA pathogen-based diagnostic tests.

51. Use of the RNA molecule selected and detected by the method of claim 32 for preparing cDNA libraries for RNA sequencing or for preparing samples for direct RNA sequencing.
